(19) United States
(12) Patent Application Publication
(10) Pub. No.: US 2011/0106036 A1
(43) Pub. Date: May 5, 2011

(54) ABSORBENT PRODUCT

(75) Inventors: Shadi Yazarloo Ståhl, Molndal (SE); Patrik Andersson, Billdal (SE); Kent Hermansson, Vastr Frolunda (SE); Elisabeth Boissier, Vallda (SE)

(73) Assignee: SCA Hygiene Products AB, Göteborg (SE)

(21) Appl. No.: 12/097,710

(22) PCT Filed: Dec. 16, 2005
(86) PCT No.: PCT/SE2005/001959

§ 371 Date: Jun. 16, 2008

(51) Int. Cl. A61F 13/512

(52) U.S. Cl. 604/383

(57) ABSTRACT

The invention refers to an absorbent product including a liquid permeable apertured top sheet facing the wearer during use, and a liquid-impermeable back sheet facing away from the wearer during use, whereby longitudinally extending textile-like edges are positioned on both longitudinal sides of the top sheet whereby the textile-like edges comprise apertures. Hereby, the textile-like edges will amongst others have the capacity to transport moisture that is formed at the interface between the textile-like edges and the wearer skin. Further, the apertures will allow a certain acquisition, i.e., liquid can be transported to the absorbent structure of the product.

### CROSS-REFERENCE TO PRIOR APPLICATION

This application claims priority to Application No. PCT/SE2005/001959 filed Dec. 16, 2005, which is incorporated by reference herein.

### FIELD OF THE INVENTION

The invention refers to an absorbent product, especially an incontinence protection, a sanitary napkin or a panty liner including a liquid permeable apertured top sheet including facing the wearer, a liquid impermeable back sheet facing away from the wearer, and an absorbent structure positioned between the top sheet and the back sheet, and optionally a liquid distribution layer between the top sheet and the absorbent structure, wherein the top sheet and the back sheet are sealed together at the periphery of the product thereby forming an edge sealing, and whereby longitudinally extending textile-like edges are positioned on both longitudinal sides of the top sheet.

### BACKGROUND OF THE INVENTION

An often occurring problem with conventional absorbent products is that the outer longitudinal parts of the product may chafe against the skin of the wearer, e.g., at the upper inner side of the thighs. This causes discomfort for the wearer. To avoid this problem, it is known to cover the longitudinal edges of the top sheet with a soft material, such as a non-woven. See, e.g., WO93/09744, WO93/12745, EP-A-1016396 and EP-A-523683. The soft longitudinal edges are sometimes referred to as “textile edges” or “textile-like edges”.

Since the textile edges will cover parts of the top sheet, they need to have some characteristics so that the properties of the product are not negatively changed. Further the textile edges may add properties to the product if that is desired.

The main property of the textile edges is that they are skin-friendly. Thus, they need to be composed of a soft material. Often, a non-woven material is used. Further, since the textile edges cover parts of the top sheet, the textile edges may have the advantage of preventing rewetting of the top sheet after liquid absorption. To provide this advantage, it is important that the textile edges do not allow liquid to flow from its inner side to the side facing the wearer. Moreover, since the absorbent structure of an absorbent product often includes SAP-granules that have a small size the textile edges may prevent SAP-granules from falling out of the product. Still further, it is desired that the textile edges will have the capacity to fit tightly to the wearer's body. This can be achieved as a result of a clever choice of material.

In order to achieve the desired effects discussed above a conventional non-perforated textile edge based on a soft material is often used (see, e.g., EP-A-523683). However, hereby some disadvantages and problems arise. To start with, it is difficult to use the capacity of the entire absorbent structure, since liquid is only let into the product in the central part of the product (i.e., the part not covered by longitudinally extending textile edges). Hereby, the absorbent structure needs to be oversized or have over-capacity in relation to the size of the product, or very efficient way for acquisition of absorbed liquid needs to be provided. This is especially a problem when it is desirable to provide a small product, i.e., a product that due to its small size is economical with regard to material use, wearer comfort and wearer handling.

A further problem with a conventional textile edge is that it normally fits tightly with the skin of the wearer. Hence, any moisture that is formed between the textile edge and the wearer skin will not be transported away. Thus, discomfort for the wearer will be the result.

It is the object of the invention to provide an absorbent product having textile edges, wherein the posed problems above are solved.

### SUMMARY

This object can be achieved by an absorbent product, wherein the textile-like edges include apertures. Hereby, the textile-like edges will have the capacity to transport moisture that is formed at the interface between the textile-like edges and the wearer skin. Further, the apertures will allow a certain acquisition, i.e., liquid can be transported to the absorbent structure of the product.

In a preferred embodiment, the ratio between the diameter of the apertures of the top sheet and the diameter of the apertures of the textile-like edges is at least 1, preferably at least 2. Hereby, the apertures of the textile-like edges are small enough to not allow SAP-granules falling out of the product. In yet another preferred embodiment the ratio between the open area of the top sheet and the open area of the textile-like edges is at least 2. Hereby, the liquid transport capacity is concentrated to the area where it is needed the most, i.e., in the central part of the top sheet. Further, it is also preferred that the top sheet material includes perforations, that are positioned between the apertures of the top sheet, having a diameter in the interval of 0.05 to 1 mm, preferably in the interval from 0.1 to 0.4 mm.

In yet another preferred embodiment, a liquid distribution layer is provided between the top sheet and the absorbent structure, wherein the liquid distribution layer in a preferred variant is in the form of a high loft layer, whereby the liquid distribution layer extends to the periphery of the product so that it forms part of the edge sealing. Hereby, the product edges will become stiff, which can contribute to wearer comfort and product function. Also, liquid that has been transported through the top sheet can be distributed towards the sides of the product so that a larger part of the absorbent structure will come in direct contact with liquid; i.e., a larger part of the absorbent capacity is used. Also, the risk for SAP-granules falling out of the product is further reduced. Still further, by having the liquid distribution layer material in the edge sealing, other sealing devices can come in question, such as ultrasonic welding, since the liquid distribution layer material can comprise weldable fibers; e.g., a high loft layer including synthetic fibers that are weldable. Hereby, the sealing strength can also become stronger. Another advantage can be that the welding pattern on the surface of the textile edges or top sheet can become clearer.

Thus, in a further embodiment, the edge sealing is at least partly formed by point welding, such as ultra-sonical point welding. Hereby, a better contact is achieved between the top sheet material and the absorbent structure, since the welding also includes the step of mechanically compressing the material of the welding points (see FIG. 1 for a principal drawing comparing conventional solution with a solution of the present invention). The material compression can also be obtained by some other sealing method including mechanical compression, but if the material, for example, is sealed by an adhesive (without mechanical compression) the material may be too loosely bound to provide a good contact between the top sheet material and the absorbent structure. As a result of a better contact between the top sheet material and the absorbent structure, a more efficient absorption is achieved. Especially, it is desirable that the ratio between the thickness of the central part of the product (T) and the thickness of the edge sealing (t) is at least 3. Hereby, a good contact between the absorbent structure and the top sheet material is achieved. Further, by using a roll material (instead of a mat formed material) for the liquid distribution layer or for the absorbent core that inherently can be compressed (since it normally is in the form of a roll) further compression and thereby improved contact can be achieved.

Accordingly, the absorbent product according to the invention will have a central part (seen from the front of the product) including the absorbent core, liquid distribution layer, and a top sheet that is raised compared to the side edges of the product. In order to provide a liquid inlet at the edges that is as good as possible, the material of the edges (nonwoven textile-like edges) should have as good contact as possible to the raised central part. According to the invention, this is achieved by providing a steep slope in the material close to the upper edge of the raised central part. Thus, the fastening points of the side-nonwoven should be positioned as close to the lower edge of the raised central part as possible.

Still further, by using ultrasonic welding for sealing the edges, the welding points will achieve a hydrophobic character. When conventional welding is used, the entire edge will get a hydrophobic character (since a continuous welding seam is provided). Hence, liquid inlet is not allowed through a conventional continuous welding seam, and there is a risk for leakage if the wearer of the product has a major liquid outlet of, e.g., urine; i.e., if the received liquid amount is so big that the top sheet material and the textile edges can not take care of all liquid. This is especially a problem if the product in question is small in size. This problem is reduced if only discrete welding points are hydrophobic, since the area between the welding points will have the capacity to transport liquid.

Moreover, in a preferred embodiment the product has a longitudinal length that is in the interval from 13 to 22 cm, preferably shorter than 15 cm, and a lateral width, at the least wide position of the crotch part, that is in the interval from 4 to 6.5 cm, preferably less than 5 cm.

One important aspect of the invention is that the product of the invention has a high absorption capacity in relation to its size. In order to achieve this advantage, some properties should be shown by the product.

To start with, the liquid inlet materials (top sheet and liquid distribution layer) should be able to take care of a relatively large volume of liquid. This is achieved by using a top sheet material having funnel-shaped three-dimensional apertures, and by providing a bulky material, such as a high loft layer, beneath the top sheet. The three-dimensional structure of the perforated material together with the high loft layer gives a high free volume (void volume) that quickly can take care of the liquid. The funnel-shaped apertures will also “collect” the liquid initially so that it does not flow off the surface. The film of the top sheet material can also be pre-perforated with small apertures (also referred to as perforations), so that the liquid also partly can be taken in between the large apertures.

Secondly, the inlet materials should be able to keep the liquid that has been taken up by the product during use. This is achieved by using the film of the top sheet material as a barrier against rewetting, i.e., to make it difficult for the liquid to return to the skin of the wearer. The SAP of the absorbent core has also, of course, a major contribution to keeping the absorbed liquid in the product. The three-dimensional structure of the top sheet and liquid distribution layer, preferably high loft layer, also makes the distance between the skin of the wearer and the core of the product longer, so that the product is experienced as drier.

Moreover, as a third point, the inlet materials need to provide a dry surface for the comfort of the wearer. This is achieved by ultra-sonically welding the top sheet material and the liquid distribution layer, preferably the high loft layer. Hereby, the top sheet material and liquid distribution layer can be properly drained.

Further, as a fourth point, the inlet materials should be soft and airy for the comfort of the wearer. This is achieved by that the material for the textile edges is made in a soft nonwoven for providing good comfort, and by that it includes perforations for being “airier” and for giving a good liquid inlet.

Thus, the invention is further directed to an absorbent product wherein the product has at least one of the following characteristics (in accordance with the experimental results shown in the example section): (i) an acquisition time for a first inlet of 5 ml synthetic urine that is below 6 s, preferably equal to or less than 5 s, and an acquisition time for a second inlet of 5 ml synthetic urine that is below 8 s, preferably equal to or below 4 s; (ii) a DORUP (retention) value for a dosage of 7 ml synthetic urine that is below 1 g, preferably below 0.5 g and more preferably equal to or below 0.2 g; or (iii) a Rothwell (Absorption capacity) value of more than 45 g, preferably more than 55 g.

### BRIEF DESCRIPTION OF THE DRAWINGS

FIG. 1 discloses a top plan view of a panty liner of the invention having asymmetric shape including apertured textile-like edges.

FIG. 2 discloses a top plan view of another panty liner of the invention including apertured textile-like edges.

FIG. 3 shows a cross-sectional view of various layers of an absorbent product of the invention.

FIG. 4 is a principal drawing comparing a conventional solution (61) with a solution of the present invention (60) with regard to contact between the top sheet and absorbent structure.

FIGS. 5-7 show experimental diagrams referring to the Example section of the invention.

### DETAILED DESCRIPTION OF THE PREFERRED EMBODIMENTS

The present invention pertains to an absorbent product. By an “absorbent product” is meant a product such as an incontinence protection, a sanitary napkin and a panty liner. The following definitions are provided below for further clarity regarding features of the present invention.

By “open area” is meant the percentage of the surface of a material that is composed of perforations or apertures.

By a “longitudinal” direction or “machine direction” is meant the direction along the length of the absorbent product, i.e., from the rear to the front of the product (or vice versa), and by “lateral” direction or “cross-direction” is meant the direction from side edge to side edge of the product, i.e., across the width of the product.

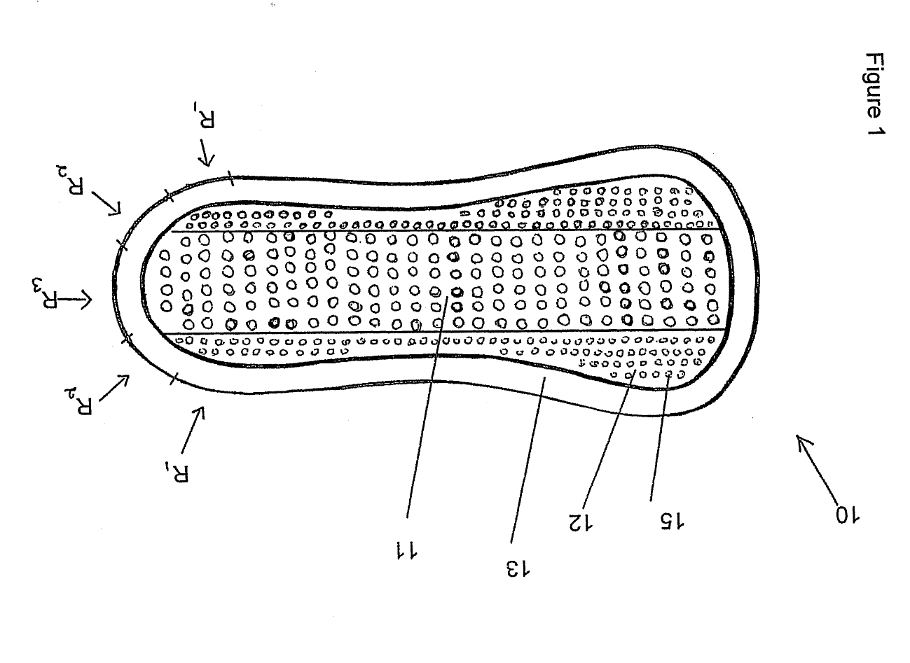

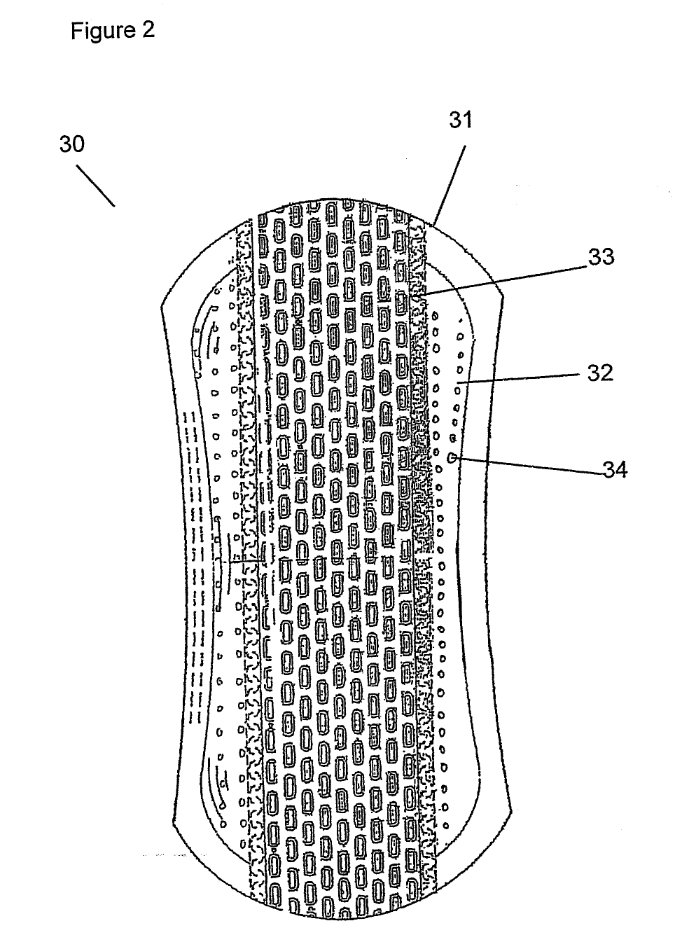

FIGS. 1 and 2 discloses absorbent products of the invention in the form of a panty liner (10) and a pad (30). As can be seen, the products are equipped with an apertured top sheet (11, 33). At the longitudinal edges of the top sheet, textile-like edges (12, 32) are provided, which textile-like edges are provided with apertures (15, 34). Further, the textile-like edges can be equipped with an embossed pattern, in order to add function or for design purposes. At the periphery of the product, an edge sealing can be seen (13, 31), which seals the various layers of the product. In a preferred embodiment, the edge sealing includes a pattern of discrete welding points, e.g., created by ultrasonic welding.

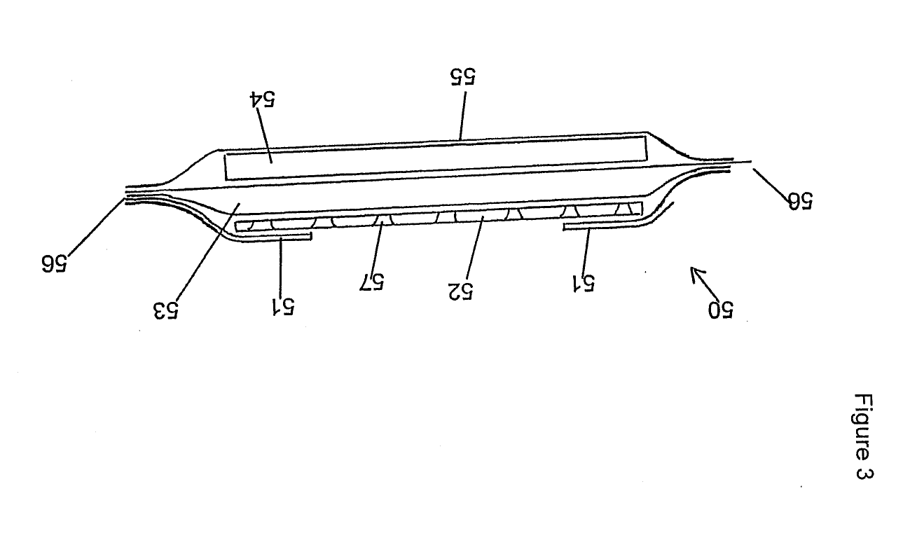

Turning to FIG. 3, a principal drawing of the various layers of the product (50) can be seen. Starting from the top (intended to be closest to the skin of the wearer at use) the textile-like edges (51) can be seen, which are positioned at the longitudinal edges of the top sheet (52). The textile-like edges can be fastened to the top sheet by way of embossing the textile-like edges, by ultrasonic bonding, by using an adhesive, such as a hotmelt, or a combination of fastening devices. Beneath the top sheet an optional liquid distribution layer (53) can be positioned. In a preferred embodiment, the liquid distribution layer is a high loft layer. Beneath the liquid distribution layer, or directly beneath the top sheet (in case no liquid distribution layer is present), the absorbent structure (54) is positioned. For example, the absorbent structure is a pressure-bonded airlaid core including superabsorbent polymers. In a preferred embodiment, in order to provide a high absorption capacity, which is necessary for a small-sized product of this type, the absorbent structure includes about 50-60% SAP. Beneath the absorbent structure, a back sheet (55) is provided. For example, the back sheet is a plastic film. Preferably, the plastic film is breathable. At the outside of the back sheet, a release paper is positioned which is fastened to the back sheet, e.g., by glue strings or by any other conventional devices. An edge sealing (56), binding the textile edges, e.g., by way of ultra-sonic welding, the liquid distribution layer and the back sheet at the longitudinal edges can also be seen. Reference numeral 57 refers to funnel-shaped apertures according to a preferred embodiment.

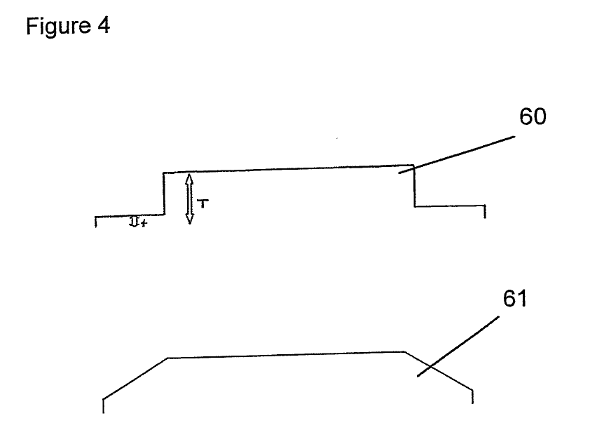

FIG. 4 shows the principles of the effect of the ultrasonic bonding of the invention. This drawing should only be interpreted schematically. As can be seen from FIG. 4 (which figure should be interpreted for principal purposes) the solution according to the invention (60) provides a more distinct contact between the top sheet and the absorbent structure. Hence the absorbent structure and the liquid distribution layer are well defined. In a conventional solution (61), the top sheet will not lie as close to the absorbent structure and the liquid distribution layer as in the present invention, which will affect absorbent and rewetting properties as discussed above.

The present invention is mainly directed to absorbent products such as an incontinence protection, a sanitary napkin or a panty liner. Each product includes a rear part, a front part and a crotch part positioned there between.

The product can be hour-glass shaped (wherein the rear part and front part essentially equally wide measured in the cross-direction, and the crotch part is less wide than the front or rear part) or it can be asymmetrically shaped or it can have any other shape that is appropriate for products of this type. By asymmetrical can be meant that the product is least wide in the crotch part of the product (measured in cross-direction) and it is wider in the front part than in the rear part (measured at the widest position of the rear and front part respectively in cross-direction). For instance, the width in the crotch part of the product can be from 4-7 cm, especially from 4.5 to 5 cm, for example about 4.7 cm. The width at the widest position of the front part can be from 5 to 8 cm, especially from 6-7 cm, for example about 6.5 cm. The width at the widest position of the rear part can be from 4.5 to 6.5 cm, especially from 5 to 6 cm, for example 5.3 cm.

In one preferred embodiment, the product of the invention is a panty-liner having a length in the interval from 13 to 22 cm, preferably of about 15.2 cm and a width at the crotch part of the product in the interval from 4 to 6 cm, preferably of about 4.7 cm. The top sheet material is a three-dimensional pre-perforated film which is equipped with further apertures, typically 7-9 apertures per row. At the edges of the top sheet, an apertured textile edge is provided that is composed of nonwoven material. A high loft layer is included as a liquid distribution layer. Further, the absorbent core is provided in the form of a roll-material including about 40-60% SAP. The edges of the product including the liquid distribution layer are sealed by way of ultra sonic bonding. The product further includes a back sheet, facing away from the wearer.

In another preferred embodiment, the invention refers to a panty liner including a carded nonwoven as a top sheet material. At the edges of the top sheet, an apertured textile edge is provided that is composed of nonwoven material. The liquid distribution layer is composed of an airlaid material and the absorbent core includes pulp and SAP. The edges of the product including the liquid distribution layer are sealed by way of ultra sonic bonding. The product further includes a back sheet, facing away from the wearer.

In still another preferred embodiment, the invention refers to a sanitary napkin including an apertured top sheet material. At the edges of the top sheet an apertured textile edge is provided that is composed of nonwoven material. Further, the sanitary napkin includes a liquid distribution layer and an absorbent core. The edges of the product including the liquid distribution layer are sealed by way of ultra sonic bonding. The product further includes a back sheet, facing away from the wearer.

In yet another preferred embodiment, the invention refers to an incontinence product or incontinence guard including a liquid pervious top sheet facing the wearer during use, optionally a liquid distribution layer underneath the top sheet, an absorbent core, and a liquid impermeable back sheet facing away from the wearer. Normally, the absorbent core includes a compressed mixed or layered structure of cellulosic fluff pulp and superabsorbent polymers, however further or other material combinations as disclosed below are fully possible. Also, instead of being provided in one layer, the absorbent core may include two separate layers, or more.

The shape of the products of the invention can be hour-glass shaped (i.e., being less wide in the central part compared to the front and rear parts, which are equally wide). Also, the products of the invention can be asymmetrically shaped, whereby the product is less wide in the central part compared to the front and rear parts, and whereby the rear part is less wide than the front part. Other shapes are also fully possible for the products of the invention.

The textile-like edges are preferably made by a soft, skin-friendly material, such as a nonwoven, so that any chafing against the skin of the wearer is reduced as much as possible. By “soft” is meant that the material is experienced as soft by the consumer. For example, the material of the textile-like edges can be a carded nonwoven. Further, the textile-like edges includes apertures.

The apertures of the textile edges can be made by mechanical perforation (e.g., a roller with hot or cold pins). Water jets or vacuum and heat or a combination of these techniques could also be used for the perforation.

The textile edges can be made of polypropylene carded thermobonded hydrophilic nonwoven (fibrous) material. Also, spunbond nonwoven, an air-thru bonded nonwoven, a spunlaced (hydroentangled) nonwoven, a meltblown nonwoven, or a combination of these can be used. If a combination is used, there can be a mixture of fibers from different polymers, but each fiber can also include different polymers (For example: bicomponent fibers PP/PE or copolymer PP/PE). The textile edge nonwoven can also include a percentage of natural fibers, such as pulp or viscose. The nonwoven can be hydrophilic, permanent hydrophilic or hydrophobic. The nonwoven can have a basis weight of 7 to 50 μm.

Also, the material in the textile edges can be a plastic film, made of PP, PE, PET, PLA, starch or any other thermoplastic polymer, or a blend or a copolymer of the polymers mentioned.

The material can also be a laminate of a nonwoven and a film. Such a laminate can be made by bonding the nonwoven and the film by using heat, by using an adhesive, by mechanical bonding or by extrusion of the film on the nonwoven, or a combination of these methods.

The textile edges preferably have an aperture size in the interval from 0.8 to 1.2 mm, preferably of about 1 mm in cross-direction (lateral direction) and in the interval from 1.2 to 1.8 mm, preferably about 1.5 mm in machine-direction (longitudinal direction). However, the aperture size can vary from about 0.2 to 6 mm in diameter.

The apertures of the textile edges are typically oval-shaped, slightly elongated in the machine direction. However, the apertures can be round/circular and or oval in both machine- and cross-direction. The density of apertures in the textile edges will normally be in the interval from 4-250/cm², preferably from 50-120/cm². Further, the apertures of the textile edges can be positioned in one or more than one longitudinal or lateral rows, or they can be positioned in a longitudinally extended row. Further the apertures can have varying sizes and forms. They can also be concentrated (zoned) to one or more zones of the textile edge, such as close to the crotch area or the expected wetting point, so that the function of the apertures is concentrated to positions where it is especially desired.

The open area of the textile edges is in the interval from 2 to 50%, more preferably in the interval from 5-20%.

The liquid-permeable top sheet is preferably made of a material showing properties like dryness and softness at use of the absorbent product, as this sheet lies against the body of the wearer. It is desired, that the sheet has a soft and textile-like surface, which remains dry also at repeated wettings. The top sheet may for example be composed of nonwoven material with a soft and smooth surface, such as for example a spunbond made of polypropylene fibers. In order to keep the surface closest to the skin of the wearer dry, a hydrophobic nonwoven-material may be used, which has apertures, so that openings are formed in the material, which openings are greater than the cavities between the fibers of the material. In this way, fluid may be lead down through the holed openings in the top sheet to the underlying absorption core. Other examples of material in the top sheet may for example be holed plastic films, such as for example a holed polyethylene film. The top sheet may be connected to the underlying back sheet and to the absorption core by, for example, glue, ultra-sonic bonding or through some kind of thermal bonding. Preferably, the top sheet is an apertured nonwoven, having an aperture density of 3-15, preferably 6-12 and more preferably 7-9 apertures/cm².

In a preferred embodiment the top sheet further includes small perforations that are positioned in the material that surrounds the apertures of the top sheet, in order to further increase the liquid inlet and acquisition properties. Normally the perforations of the top sheet have a density in the interval of 20-500, preferably 70-250 and more preferably 120-170 perforations/cm².

It is desirable that the caliper value of the top sheet material is as high as possible, since a high caliper value has a beneficial effect on the acquisition. However, in order to not affect the softness of the material negatively, a balance needs to be obtained, and thus a caliper value in the interval from 1.3 to 1.7 mm, preferably about 1.5 mm, has shown to be adequate.

Also, the top sheet material can be a three-dimensional laminate of nonwoven and plastic film. The nonwoven can be a carded thermobonded 100%-polypropylene based, hydrophilic material. The plastic film can be hydrophilic, pre-perforated (small holes), and made of a blend of polyethylene and polypropylene.

Also, the nonwoven part of the top sheet material can be a spunbond nonwoven, an air-thru bonded nonwoven, a spunlaced (hydroentangled) nonwoven, a meltblown nonwoven, or a combination of these. Raw material can be polypropylene (PP), polyethylene (PE), polyester (PET), polyamide (PA), or a combination of these. If there is a combination, there can be a mixture of fibers from different polymers, but each fiber can also include different polymers (for example, bicomponent fibers PP/PE or copolymer PP/PE). The textile edge nonwoven can also include a percentage of natural fibers, such as pulp or viscose. The nonwoven can be hydrophilic, permanent hydrophilic or hydrophobic. The nonwoven can have a basis weight in the interval from 7 to 50 μm.

The plastic film can also be made of PE or PP, PET, PLA or starch (or any other thermoplastic polymer), or a blend or copolymer of the polymers mentioned.

The perforated top sheet can also be made of a single layer material, such as a nonwoven or film (as described above).

The top sheet can have an aperture size of 1.6 to 3.2 mm in the machine direction (longitudinally) and 0.9 to 2.3 mm in the cross direction (laterally). The aperture size can be from 0.1 to 6.0 mm in diameter.

The apertures of the top sheet can be oval-shaped, slightly elongated in the machine direction. The apertures can be round/circular, or oval in machine direction or cross direction.

The open area of the top sheet can be in the interval from 2 to 60%, preferably from 5-30%, more preferably from 10-20%, most preferably about 14%.

The liquid-impermeable back sheet includes a flexible material, preferably a thin plastic film of PE (polyethylene), PP (polypropylene), a polyester, or some other kind of suitable material, such as a hydrophobic nonwoven-layer or a laminate of a thin film and a nonwoven material. These types of laminates are often used in order to achieve a soft and a textile-like surface of the back sheet. In order to accomplish an airier and comfortable product, it is also possible to use breathable back sheets, which prevents fluid from coming out of the absorbent product, but that allows moisture to be ventilated. These breathable back sheets may be composed of single material layers, or of laminates of, for example, blown or moulded polyethylene films, which have been laminated with, for example, a nonwoven layer of spunbond or of spunbond-meltblown-spunbond (SMS).

The absorbent structure is typically built up by one or more layers of cellulose fibers, for example cellulose fluff pulp. Other materials, which may be used, are for example absorbing nonwoven material, foam material, synthetic fibre materials or peat. In addition to cellulose fibers or other absorbing materials, the absorbent structure may also include superabsorbent material, so called SAP (super absorbent polymers), that is material in the form of fibers, particles, granula, film or the like, which material has the ability to absorb fluid corresponding to several times the weight of the superabsorbent material. The superabsorbent material binds the fluid and forms a fluid-containing gel. Moreover, the absorbent structure may include binders, form-stabilizing components or the like. The absorbent structure may be chemically or physically treated in order to change the absorption properties. For instance, it is possible to provide an absorbent layer with compressed regions and/or being compressed in the entire layer(s) in order to control the fluid flow in the absorbent body. It is also possible to enclose the absorbent layer(s) in an envelope of for example tissue material. For example, the absorbent structure is an airlaid, pressure-bonded structure including 30-80%, preferably about 40-60%, more preferably 50-60% SAP.

Typically, the absorbent structure has in its longitudinal direction an outstretched form, and may for example be essentially rectangular, T-shaped or hourglass-shaped. An hourglass-shaped absorbent body is wider in the front and rear parts than in the crotch part, in order to provide an efficient fluid absorption simultaneously as the design facilitates the product to form and to close around the user, thereby giving a better fit around the legs. In a preferred embodiment of the present invention, the absorbent structure has essentially straight and parallel longitudinal edges. The rear and front edges are rounded in order to fit the shape of the product. Preferably, the rear and front edges are rounded in the form of at least three different radii.

Also, the absorbent structure can be provided in the form of a roll-material, whereby the material typically is provided with a high compression, or the structure can be formed by way of air-laying technique.

In yet another embodiment, the absorbent structure is equipped with a wicking layer, which wicking layer has the purpose to spread fluid towards the front part of the absorbent structure. Moreover, the wicking layer does not necessarily need to cover the whole absorbent structure, but should preferably cover at least the part of the absorbent structure being in the front part of the casing, more preferably the part being in the front and crotch parts of the casing, and most preferably the entire absorbent structure.

The wicking layer is of a moisture permeable material, preferably tissue paper or a hydrophilic non-woven, and functions to disperse the fluid, i.e. urine, passing through the liquid permeable top sheet, preferably in a direction towards the front part of the diaper. The wicking layer includes small capillaries directing the fluid towards smaller capillaries, due to capillary forces.

Additional liquid distribution layers may be used in the product of the invention, preferably between the absorbent structure and the top sheet. For instance, additional layers improving the properties may be used, such as a transfer layer or various types of fluid-spreading material layers or inserts, so called waddings or high-loft layers. Typically, the liquid distribution layer is a porous, resilient, relatively thick material layer, for example in the form of a fibrous high loft layer, a carded fibrous web, a tow material or other type of bulky and resilient fibrous material having a high momentaneous liquid receiving capacity and which can temporarily store liquid before it is absorbed by the underlying absorbent core. Also, the liquid distribution layer may be in the form of a porous foam material. Also, it may include two or more material layers. In a preferred embodiment, the liquid distribution layer extends to the side edges of the product, i.e., it has basically the form of the top sheet or the top sheet and the textile edges. Hereby, advantages with regard to liquid distribution, edge sealing etc, as discussed above, can be achieved. However, the liquid distribution layer can also be designed so that it does not extend into the edge sealings. The transfer layer can be an airlaid layer and it can include SAP.

Further, the product of the invention may be equipped with a release paper that covers the outer side of the back sheet of the product. The release paper may be fastened to the back sheet by way of glue strings.

At the periphery of the product, with a width of about 0.5 cm, an edge sealing is provided in order to secure the top sheet, the back sheet and optionally the liquid distribution layer to each other. The edge sealing can be made by way of adhering the layers to each other, by way of embossment, by way of heat welding, by way of ultra sonic bonding, or a combination of these methods. Preferably, ultra-sonic bonding is used. Also, the sealing can be made as a continuous sealing or as discrete point weldings. Preferably, the sealing is made as point weldings, since this provides some advantages with regard to, e.g., hydrophobic character of the welding points as discussed above. Typically, the welding points are at a distance of about 0.1-0.5 mm from each other preferably about 0.2 mm. For performing the ultra-sonic bonding, an ultrasonic bonding equipment is used. When the ultra-sonic bonding is performed, a welding pattern is formed, which welding pattern can have a form that is visually attractive and/or that have technical advantages, such as an improved comfort for the wearer or effects with regard to liquid distribution or improved softness and/or dryness.

In order to prevent fluid to leak out, the absorbent product on the side that is facing the wearer may also be equipped with inner fluid barriers, which are attached in connection to the longitudinal edges. Preferably, the inner barriers are made of an essentially liquid-impermeable material, such as for example a hydrophobic nonwoven or a plastic film, and are formed as a longitudinal path with a first edge being connected to the absorbent product and a second free edge, which is adapted for being in close contact with the user at use of the absorbent product. The second edge is equipped with one or more elastic elements, preferably an elastic thread, which in contracted state contracts the free edge, whereby an upstanding barrier is formed. The inner barrier may be designed as a strip of a single sheet, wherein the free edge is turned down in order to enclose the elastic element to prevent direct contact of the elastic thread to the user. Alternatively, the barrier may be formed of two combined layers, whereby the elastic thread is attached to the edge of the free end between the two layers. In this case, the inner layer of the barrier may be composed of an elongation of the top sheet and the outer layer of an essentially liquid-impermeable material, or the inner and outer layers of the barrier may be composed of one single material strip, which is folded around the elastic thread. Further, the elastics can be provided in the form of foam, e.g., in a band or thread, or the elastics can be provided in any other conventional way.

Further, the product of the invention may be equipped with wings. Also products equipped with other layers than described in this disclosure are also included in the scope of the invention.

### EXAMPLES

In all the attached examples, properties were compared between a panty-liner product according to one embodiment of the invention (here called “Tena”) and various conventional panty-liner products (see table 1 and 2 for data for the compared products). The compared products have structures that are similar to Tena. The “Tena-product” has an asymmetric shape (see FIG. 1 for a principal drawing), the top sheet is a perforated laminate between a nonwoven material and a plastic film having a basis weight of about 40 μm having apertured top sheet that also includes small perforations. Further, the product includes textile edges, a nonwoven high loft layer (basis weight of about 50 μm) as liquid distribution layer, an absorbent core of cellulose fibers and SAP (about 50-60%) and a breathable back sheet. The “Tena” product is a small panty liner product, and thus the experimental values should be interpreted with regard to a small panty liner. However, since the results that are shown for the Tena product can be regarded as an effect of using quick inlet materials (top sheet having large apertures, textile edges having apertures, and an absorbent core having a large amount of SAP (about 50-60%)), the analogous results can be expected for other product types that are formed in a similar way by the same principles, but being longer, heavier and thicker.

TABLE 1

| Product | Weight (g) | Thickness (mm) | Length (mm) |
|---|---|---|---|
| Tena | 3.4 | 2.9 | 152 |
| Alldays Normal | 2.8 | 2.9 | 150 |
| Always Regular (U.S.) | 3.1 | 2.6 | 230 |
| Alldays Small | 1.7 | 2.8 | 142 |
| Libresse Normal | 2.9 | 2.8 | 150 |
| Carefree Original | 2.4 | 3.1 | 160 |
| Kotex Normal | 2.1 | 2.6 | 152 |
| Poise light liners | 3.3 | 2.7 | 165 |
| Alldays Extra Large | 3.9 | 3.0 | 177 |
| Always Super Long (U.S.) | 4.1 | 3.1 | 280 |
| Carefree Maxi Large | 3.0 | 3.0 | 180 |
| Lindor active mini Ultra | 3.8 | 2.3 | 165 |
| Libresse Large | 3.8 | 2.6 | 175 |

TABLE 2

| | | Carefree | Libresse | Kotex | Alldays | Alldays | Alldays |
|---|---|---|---|---|---|---|---|
| Construction | Tena | Original | Normal | Normal | Small | Normal | Extra Large |
| Surface | laminate | Thermobound | Carded | Nw | 3D-PE | 3D-PE | 3D-PE |
| | | PP-nw | nw | “Coform” | “cotton- | “cotton- | “cotton- |
| | | | | | like” | like” | like” |
| Drainage | High loft | Thermobound | Airlaid | no | Thermo- | Thermo- | Thermo- |
| | layer | bicomponent | | | bound | bound | bound |
| | | PP/PE | | | PP-nw | PP-nw | PP-nw |
| Absorption | Novathin, | Novathin, | Pulp and | Airlaid | Airlaid | 2-layer | 2-layer |
| material | SAP | airlaid | SAP | pulp- | with SAP- | airlaid with | airlaid with |
| | | with SAP | | system | fibers | SAP | SAP |
| SAP (% of | About 55 | — | About 8 | no | — | About 24 | About 31 |
| product) | | | | | | | |

### Example 1

### Acquisition

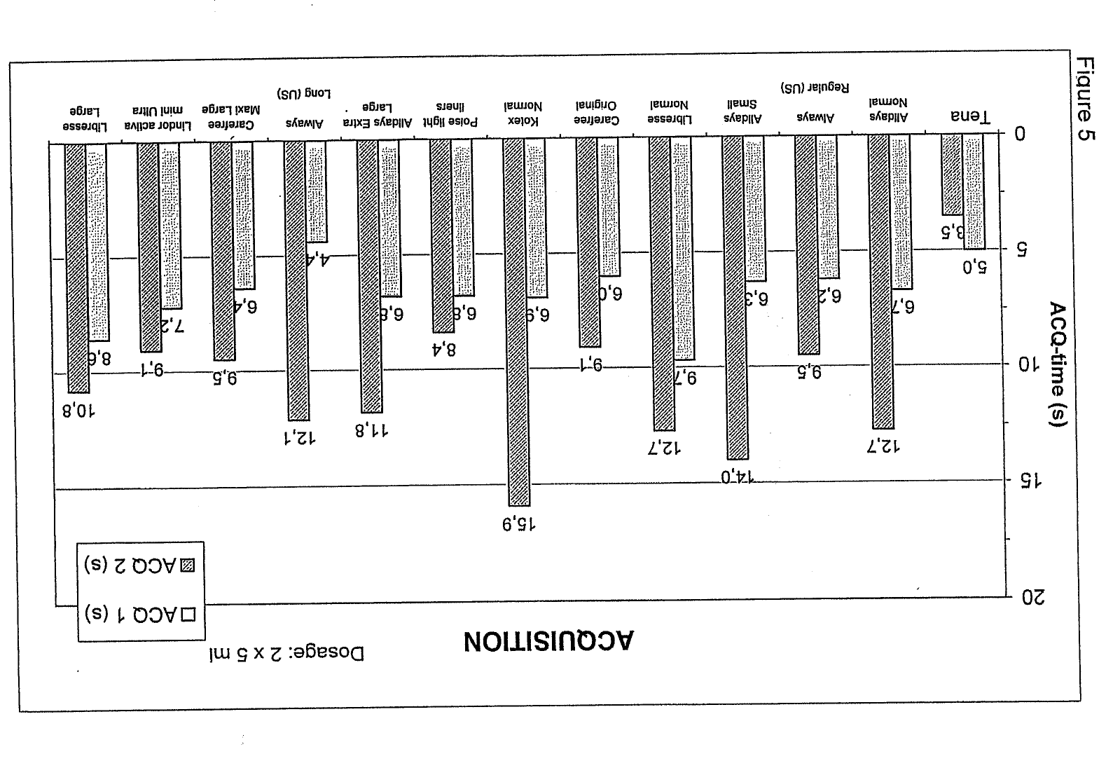

Acquisition properties (acquisition time) were tested (test method no 17-28-14) by exposing the various products for a first inlet of 5 ml synthetic urine followed by a second inlet of 5 ml synthetic urine (0.9% NaCl in water) with an interval of 10 min. The results can be seen in FIG. 5, showing that Tena is significantly faster than almost all the other products for the first inlet, with exception to Always pantiliners Long, and significantly faster than all other products for the second inlet. This shows that the construction of Tena is very capable of quick acquisition of liquid (not the least of urine type), thereby reducing risk for any leakage.

### Example 2

### DORUP (Retention)

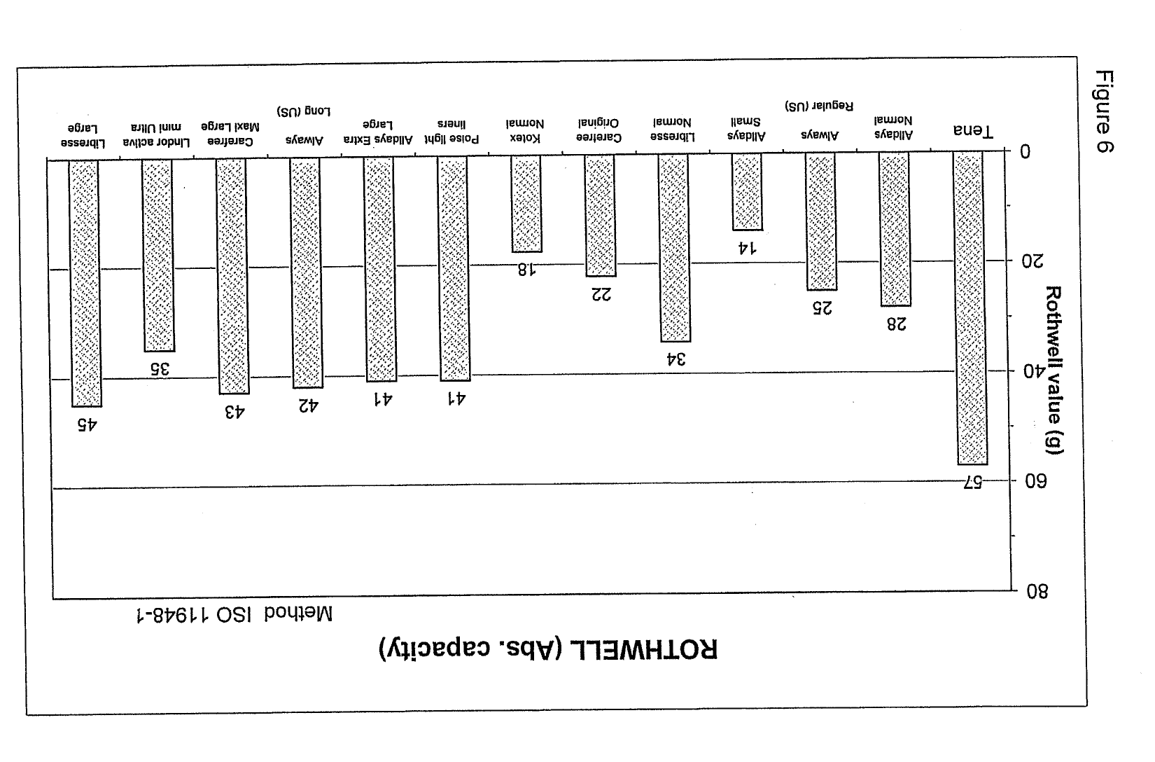

Retention properties were tested by exposing the various products for an inlet of 7 ml liquid (synthetic urine) (see FIG. 6). For more details concerning the DORUP (retention)-experiment, reference is made to U.S. Pat. No. 6,557,398, which is incorporated herein by reference. As can be seen, Tena is significantly drier than all compared products. This shows that the retention properties of Tena is superior to compared products.

### Example 3

### Rothwell (Absorption Capacity)

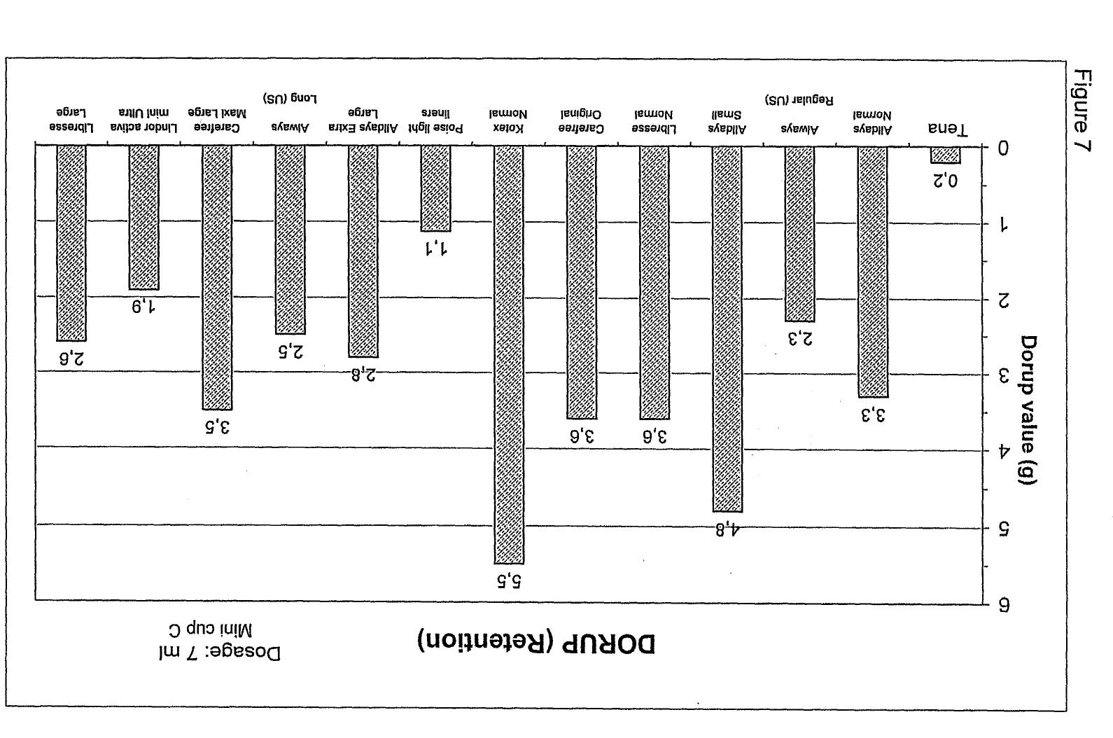

The Rothwell value (g) was tested (Method ISO 11948-1) (see FIG. 7). Tena was shown to have a significantly higher absorption capacity than all compared pantyliners.

1. An absorbent product chosen from an incontinence protection, a sanitary napkin and a panty liner, comprising:
a liquid permeable apertured top sheet facing the wearer during use, and
a liquid-impermeable back sheet facing away from the wearer during use, wherein longitudinally extending textile-like edges are positioned on both longitudinal sides of the top sheet and the textile-like edges include apertures.

2. The absorbent product according to claim 1, wherein the apertures of the textile-like edges have a diameter in the interval from 0.2 to 6 mm, a size in the cross-direction from 0.8 to 1.2 mm and a size in the machine direction from 1.2 to 1.8 mm.

3. The absorbent product according to claim 1, wherein a density of apertures in the textile edges is in the interval from 4-250 apertures/cm².

4. The absorbent product according to claim 1, wherein the apertures of the top sheet have a size in the machine-direction in the interval from 1.6 to 2.4 mm and a size in the cross-direction in the interval from 0.9 to 2.3 mm.

5. The absorbent product according to claim 1, wherein a ratio between a diameter of the apertures of the top sheet and a diameter of the apertures of the textile-like edges is at least 1.

6. The absorbent product according to claim 1, wherein a density of apertures in the top sheet is in the interval from 3-15 apertures/cm².

7. The absorbent product according to claim 1, wherein the top sheet material further comprises perforations, that are positioned between the apertures of the top sheet, having a diameter in the interval of 0.05 to 1 mm.

8. The absorbent product according to claim 7, wherein a density of perforations in the top sheet is in the interval from 30-500 perforations/cm².

9. The absorbent product according to claim 1, wherein a ratio between an open area of the top sheet and an open area of the textile-like edges is at least 2.

10. The absorbent product according to claim 1, wherein the top sheet and the back sheet are sealed together at a periphery of the product thereby forming an edge sealing, and wherein the edge sealing at least partly is formed by point welding.

11. The absorbent product according to claim 10, wherein the welding points have a hydrophobic character.

12. The absorbent product according to claim 10, further comprising a liquid distribution layer between the top sheet and the absorbent structure, whereby the liquid distribution layer extends to the periphery of the product so that it forms part of the edge sealing.

13. The absorbent product according to claim 12, wherein the liquid distribution layer is in the form of a high loft layer.

14. The absorbent product according to claim 1, wherein in a ratio between a thickness of a central part of the product (T) and a thickness of the edge sealing (t) is at least 3.

15. The absorbent product according to claim 1, wherein the product has a longitudinal length that is in the interval from 13-22 cm, and a lateral width, at the least wide position of the crotch part, that is in the interval from 4 to 6.5 cm.

16. The absorbent product according to claim 1, wherein the product has an acquisition time for a first inlet of 5 ml synthetic urine that is below 6 s, and an acquisition time for a second inlet of 5 ml synthetic urine that is below 8 s.

17. The absorbent product according to claim 1, wherein the product has a DORUP (retention) value for a dosage of 7 ml synthetic urine that is below 1 g.

18. The absorbent product according to claim 1, wherein the product has a Rothwell (Absorption capacity) value of more than 45 g.

19. The absorbent product according to claim 3, wherein the density of apertures in the textile edges is in the interval from 50-120 apertures/cm².

20. The absorbent product according to claim 5, wherein the ratio between the diameter of the apertures of the top sheet and the diameter of the apertures of the textile-like edges is at least 2.

21. The product according to claim 6, wherein the density of apertures in the top sheet is in the interval from 6-12 apertures/cm².

22. The product according to claim 6, wherein the density of apertures in the top sheet is in the interval from 7-9 apertures/cm².

23. The absorbent product according to claim 7, wherein the diameter of the perforations of the top sheet is in the interval of 0.1 to 0.4 mm.

24. The absorbent product according to claim 8, wherein the density of perforations in the top sheet is in the interval from 50-200 perforations/cm².

25. The absorbent product according to claim 8, wherein the density of perforations in the top sheet is in the interval from 70-120 perforations/cm².

26. The absorbent product according to claim 10, wherein the edge sealing at least partly is formed by sonic point welding.

27. The absorbent product according to claim 15, wherein the longitudinal length of the product is smaller than 15 cm the lateral width at the least wide position of the crotch part is smaller than 5 cm.

28. The absorbent product according to claim 16, wherein the acquisition time for the first inlet of 5 ml synthetic urine is less than 5 s and the acquisition time for the second inlet of 5 ml synthetic urine equal to or below 4 s.

29. The absorbent product according to claim 17, wherein the DORUP (retention) value for a dosage of 7 ml synthetic urine is below 0.5 g.

30. The absorbent product according to claim 17, wherein the DORUP (retention) value for a dosage of 7 ml synthetic urine is below 0.2 g.

31. The absorbent product according to claim 1, wherein the product has a Rothwell (Absorption capacity) value of more than 55 g.
